(19) United States
(12) Patent Application Publication
(10) Pub. No.: US 2023/0367388 A1
(43) Pub. Date: Nov. 16, 2023

(54) Artificial Intelligence Metrics for Quarterback Position in the National Football League

(71) Applicants: R. Kemp Massengill, Palos Verdes, CA (US); David E. Newman, Poway, CA (US)

(72) Inventors: R. Kemp Massengill, Palos Verdes, CA (US); David E. Newman, Poway, CA (US)

(21) Appl. No.: 18/356,882

(22) Filed: Jul. 21, 2023

Related U.S. Application Data
(60) Provisional application No. 63/527,665, filed on Jul. 19, 2023.
(60) Provisional application No. 63/512,984, filed on Jul. 11, 2023.
(60) Provisional application No. 63/512,729, filed on Jul. 10, 2023.
(60) Provisional application No. 63/059,074, filed on Jul. 30, 2020.

(51) Int. Cl. G06F 3/01; A61B 5/11

(52) U.S. Cl. CPC G06F 3/011; A61B 5/1118; A61B 2503/10

(57) ABSTRACT

The present disclosure shows how artificial intelligence (AI) can analyze the large number of physical, mental, and other intangible metrics - as well as analyzing all available background information - to determine which athlete in a list of potential candidates is most likely to succeed in the quarterback position in American-style football, according to the draft-candidate procedures of the National Football League. Data on each quarterback candidate’s strength, agility, speed, mental focus, and other features can be provided as input to the AI model, which then provides a prediction of the quarterback candidate’s probability of success at the next highest level, and especially on the fiercely-grueling and merciless playing field of NFL football, where failure and defeat is not an option. Additionally, the AI model can output a ranking of all the candidates at all player positions, including, of course, the truly vital position of quarterback, to further support a draft selection of potentially-worthy athletes.

### PRIORITY CLAIMS AND RELATED APPLICATIONS

This application claims the benefit of U.S. Provisional Pat. Application Serial No. 63/059,074, entitled “ARTIFICIAL INTELLIGENCE FOR NATIONAL FOOTBALL LEAGUE ATHLETIC METRICS”, filed May 25, 2023, and U.S. Provisional Pat. Application Serial No. 63/512,729, entitled “ARTIFICIAL INTELLIGENCE FOR NATIONAL FOOTBALL LEAGUE ATHLETIC METRICS”, filed Jul. 10, 2023, and U.S. Provisional Pat. Application Serial No. 63/512,984, entitled “ARTIFICIAL INTELLIGENCE FOR NATIONAL FOOTBALL LEAGUE ATHLETIC METRICS”, filed Jul. 11, 2023, and U.S. Provisional Pat. Application Serial No. 63/527,665, entitled “ARTIFICIAL INTELLIGENCE METRICS FOR QUARTERBACK POSITION IN THE NATIONAL FOOTBALL LEAGUE”, filed Jul. 19, 2023, all of which are hereby incorporated by reference in their entireties.

### FIELD OF THE INVENTION

The disclosure pertains to AI-based prediction of athletic performance, and more particularly to prediction of NFL football player performance at the quarterback position, using AI (Artificial Intelligence) models and algorithms derived from AI models.

### BACKGROUND OF THE INVENTION

Athletic analysis of potential ability at the quarterback position is currently wrought with many subjective and often-wrong decisions, thus explaining why first-round professional football quarterback picks with amazing college credentials often ‘flame out’ and are ‘busts’ in the National Football League (‘NFL’). The list of these unfortunate ‘busts’ at quarterback - by far the most important position of all positions in football - is a very long one, to the huge chagrin of NFL owners, who generally ‘guarantee’ many millions of dollars to these high draft choice quarterback flame-outs.

Consider this: The NFL pro football quarterback renowned as the Greatest of All Time (‘GOAT’) was a lowly 6th round draft pick as the number 199 player taken in the 2000 NFL draft. This is Tom Brady, who quarterbacked NFL teams to an almost unbelievable 7 Super Bowl victories - winning in 5 of these Super Bowls the Most Valuable Player Award. It should be noted that quarterback players drafted in the 6th round are generally suitable only for the ‘practice squad’ and are, essentially, ‘cannon fodder’ in the jargon of professional football.

And it is a fact that six quarterbacks were drafted before Tom Brady in the 2000 NFL draft. What happened to these quarterbacks:

The six quarterbacks drafted by the NFL before Brady combined to start 191 games and throw 258 touchdowns. Brady WON 286 games in his career, including seven Super Bowls, and threw 737 touchdowns. None of the six quarterbacks is remembered very much today, except by very hard-core football fans.

To accentuate how super-human Tom Brady became in his NFL career, and certainly to the amazement of everyone - and especially to the coaches and scouts who evaluated him as an NFL pro football quarterback prospect and drafted him as the lowly 199 pick - Brady finally retired at the truly remarkable age of 45 - when many of his teammates were in their 20s (to ultimately retire before they were 30). Tom Brady was old enough to be the father of many of his wide receivers, for instance.

For completeness, the next highest NFL Super Bowl winners are Terry Bradshaw and Joe Montana -with 4 Super Bowl victories apiece.

For the record, Terry Bradshaw was selected out of Louisiana Tech University as the Number 1 pick in the first round of the 1970 NFL draft (they got it right on this one); and Joe Montana from Notre Dame was selected at the very bottom of the 3rd round in the 1979 NFL draft as pick number 82. Both Terry Bradshaw and Joe Montana are enshrined in the NFL Pro Football Hall of Fame (as will, of course, be Tom Brady).

But how can you explain the analytical difference between the NFL quarterback draft selections of Terry Bradshaw (Number 1), Joe Montana (Number 82), and Tom Brady (Number 199) - and the six quarterbacks drafted by the NFL ahead of Tom Brady? With the current art, these huge discrepancies simply cannot be explained away. The current art of quarterback talent evaluations in the National Football League often fails, and sometimes fails miserably.

Here is another example where the NFL talent-appraising ‘experts’ (scouts/coaches/owners, etc.) fell woefully short of the mark in their analyses of two top-tier college quarterback prospects: Peyton Manning and Ryan Leaf. The vast differences in ultimate performance of these top two NFL first-round draft choices is baffling beyond explanation.

The facts are that Peyton Manning was drafted in the 1998 NFL draft as the first-round Number 1 pick, and Ryan Leaf was drafted as the NFL first-round Number 2 pick. Before a single down of NFL football, the consensus was that both quarterbacks were essentially of the same ultra-premium caliber. But what happened?

Peyton Manning won two NFL Super Bowls and is in the NFL Pro Football Hall of Fame. Ryan Leaf was unsuccessful in the NFL, and after several personal setbacks, became a football analyst and motivational speaker. Why the difference?

In signing an expensive draftee - or, for that matter, a free-agent NFL quarterback who proves to be a bust - the team has another disaster to contend with, and that is the ‘hit’ against the team’s salary cap. Currently set at $224.8 million for each team per year, if a quarterback who fails to produce chews up, say, $30 million of that salary cap, this leaves less available to buy elite athletes at other positions to somehow, in some way, rescue the season by compensating for the huge fiasco of having the chosen potential Super-Bowl-caliber quarterback not able to answer the bell. It is all but impossible to win the Super Bowl by having only the 2nd, 3rd, and 4th-string quarterbacks. So, what happens? Another year mired in ho-hum mediocrity. Another year of disgruntled fans. Another year where the thrill and the joy of a Super Bowl victory evaporate. No Holy Grail - only the bitter plum of losing. What might have been, could have been, should have been.... gone with the hopes and aspirations of totally frustrated owners and coaches, and most certainly millions upon millions of die-hard fans. And for the losing teams, the off-season is very, very long, indeed.

And here is yet another disaster when an elite quarterback draftee does not pan out. And that is the team drafting this elite college quarterback often has to ‘trade up’ in the NFL draft by forfeiting valuable draft choices to ‘acquire’ the draft choice high enough to facilitate obtaining this quarterback. So, that’s three disasters: The First disaster: the quarterback is a bust and not the hero lifesaver as sincerely hoped and prayed for. The Second disaster: the ‘hit’ against the salary cap, as described above. And now comes the Third disaster: the forfeited draft choices, which could have filled other positions of need, are now gone with the winter wind blowing off the icy Great Lakes. Again, that’s three 3 huge disasters - enough disasters to have a large jar of Tum’s on the left side of the desk, and a large bottle of Tylenol on the right side....

And even at a lower level, colleges with ‘fanatical’ National Championship aspirations - such as most certainly Alabama, Clemson, Georgia, USC, Ohio State, LSU, Notre Dame, and so on - also want answers as they rate top high school quarterback prospects. Of note is that coaches can get fired even with winning seasons at some prestigious schools. The point is this: Having a second-rate quarterback at ANY level of play - high school, college, or the NFL - usually spells doom for that team.

The essential and obviously very serious - and often very expensive -conundrum with the current prior art for NFL quarterback draft section is that no one currently knows definitively and in advance of actual on-the-field playing time how such-and-such a top quarterback prospect will actually perform when under fire, so to speak, at the next highest level. And there is no higher level that the National Football League where WINNING is the ONLY thing. Nothing else matters in the world of NFL football.

Thus, the question extremely vital for a successful NFL quarterback draft pick is this: Can something be done to mitigate the very real possibility - even the likelihood, as it frequently turns out - of a ‘bust,’ in view of the fact that there are so many very expensive NFL high draft-pick quarterback ‘busts’? National Football League owners whose pocketbooks are frequently burned to the tune of millions of dollars, or even hundreds of millions, desperately desire to have the answer to this question.

This Background is provided to introduce a brief context for the Summary and Detailed Description that follow. This Background is not intended to be an aid in determining the scope of the claimed subject matter nor be viewed as limiting the claimed subject matter to implementations that solve any or all of the disadvantages or problems presented above.

### SUMMARY OF THE INVENTION

The position of quarterback in the National Football league (NFL) is the most difficult of ANY position in ANY sport to determine in advance of actual NFL playing time how well a drafted quarterback will perform in the NFL.

It is true that coaches and scouts who analyze potential NFL athletic talent at every position attempt to consider anything and everything which is believed to influence a prospect’s ultimate athletic performance in the NFL.

But weighing the relative, and often very subtle importance of these numerous variables (‘metrics’) from one potential NFL quarterback prospect to another is difficult for human beings, who have their own backgrounds, and their own personal biases, both of which can definitely influence the final yes-draft-very-high / no-don’t-draft-very-high decision / no-don’t-draft-at-all decision for a particular athlete.

And the same can be said of other positions in football at the NFL level, although not nearly to the same degree of performance uncertainty as with the position of quarterback. With quarterbacks, it is truly an enigma who will perform in the NFL at the hoped for level, and who will be an expensive bust.

Yes, the use of computers has certainly become helpful in analyzing athletic ability - such as in baseball, a sport in which the metrics are quite basic and easy to computerize (batting average, pitcher velocity, pitcher accuracy, ability to get ‘hits’ with men on base, etc.).

And it is a fact that many professional Hall of Fame major-league baseball players were ‘phenoms’ in their teenage years, and it was obvious that huge success would be their destinies. Why was it obvious?

For instance, if a pitcher in college or high school can consistently throw at 90 miles-an-hour, and even faster - AND CAN THROW STRIKES AT WILL, and has a good curve ball and/or sinker, and is a durable, big-strong kid, it does not take artificial intelligence, or a psychic mind-reader with Tarot cards. to determine that this athlete is a solid major-league baseball prospect - and most likely not a bust. A pitch velocity analyzer and a pencil and paper, or a hand-held calculator or laptop computer, in the hands of a seasoned scout in the stands is all that is really required. And likewise, if an otherwise excellent hitter at a lower level has trouble with curve balls and other breaking pitches, forget it! He is not going to be a major-leaguer, and the only way he will watch big-league games is on television or by purchasing a ticket.

For further comparisons, now let’s consider other sports, as we have above for baseball, such as tennis and golf.

Tennis champion Roger Federer won 4 junior singles titles while a teenager, including the Junior Wimbledon championship. It was obvious that he was destined for greatness. In his phenomenal career, Federer went on to win 104 singles titles and 20 Grand Slams titles. For all practical purposes, he is said to have ‘owned’ Wimbledon, with 8 singles Wimbledon Grand Slam titles.

Next, let’s look at golf: Tiger Woods won 3 U.S. Junior championships, and as Federer dominated professional tennis, Tiger Woods went on to dominate professional golf. From a young age, greatness was his destiny.

In these sports - tennis, golf, baseball, and others - the use of computers, while helpful, is not essential for evaluating the talent of a youngster destined for the big-time professional arena. The addition of artificial intelligence in these sports, while importing ‘sizzle’, does not improve the prediction accuracy to any drastic degree. The talent is obvious for even a novice sports fan to witness. Fame awaits these youngsters!

But for the position of NFL quarterback, the calculus is very different and far more complex than in baseball, or any other sport, for that matter. Why? Because there are so many, many often very subtle variables at the position of quarterback - more variables in fact than at ANY other position in ANY other sport -and that includes other football positions. And nowhere is this more evident than at the very highest level - namely, the National Football League.

There is something peculiar and uniquely different about the position of National Football quarterback when compared to the quarterback position at lower levels.

For instance, wide receivers in college who are top-tier athletes generally continue to perform as top-tier athletes in the NFL. Likewise, linemen, defensive backs, and running backs who are stars at the college level are very likely to continue to be stars in the NFL - and, in fact, are often selected as “All-Pro” National Football League stars. Why is this not the case with NFL quarterbacks? It is because for linemen, wide receivers, defensive backs, and running backs, the college football game is essentially similar to the professional football game, except that the athletes are better and faster.

Yes, artificial intelligence can certainly be helpful in evaluating NFL positions other than quarterback. But the decision making is easier and less convoluted for these other positions. This is because with positions other than quarterback, the athletes are playing the same game at the college level and at the NFL level.

This is NOT the case with the position of NFL quarterback. Why is this?

The position of NFL quarterback is hugely different for any other position in football. For instance, the college quarterback and the NFL quarterback are, in essence, playing two different positions. It is akin to chess and checkers. Both utilize the same playing board, but are vastly different games. Playing quarterback at the college level is, so to speak, playing the simpler game of checkers, while playing quarterback at the NFL level is playing chess, a much more difficult and complex game to master.

Decisions at the NFL quarterback level must be made with much less time for thinking and deliberation than what is available for such in the college football game. Multiple options must be instantaneously processed by an NFL quarterback. And windows of opportunity, such as an open receiver, close fast, usually in milliseconds! There is no luxury - such as ‘let me mull this over for a while’ - in the NFL. And wrong decisions by an NFL quarterback are instantly punished by the uniformly outstanding athletes on the defensive side of the ball. It is a fact that there no ‘bad’ players on any NFL defensive squad.

So, what can greatly improve the accuracy of predicting quarterback winners at the NFL level, based upon analysis of quarterback winners at the college level, and upon analysis of the gigantic number of individual metrics which can affect on-the-field quarterback performance?

Again, for the position of quarterback at the college level and at the NFL level, these are two very different games. To the deep consternation of NFL owners and coaches, the talents of a college star quarterback may not, and often do not, translate into success in the NFL. Currently, much ‘guesswork’ is involved in drafting college quarterbacks into the NFL, with a plethora of subjective decision making.

And there is no question that costly quarterback draft failures are the very worst disaster for an NFL team. For the position of NFL quarterback, the draft can be an unfathomable morass, a dank swamp where failures abound - often a bridge too far.

But there is hope: The salvation for this quarterback NFL draft conundrum is artificial intelligence, which sifts through a gargantuan number of variable metrics to determine future NFL greatness. And why is this important?

Very simply, regardless of the talent of the football players on a NFL team other than quarterback, the Holy Grail of winning the Super Bowl is impossible without a truly fantastic quarterback. This is why quarterback Patrick Mahomes of the Kansas City Chiefs and winner of 2 Super Bowls recently signed a long-term contract for $450 million dollars, and this is why quarterback Lamar Jackson, considered capable of winning Super Bowls for the Baltimore Ravens, just signed a $260 million-dollar year contract. These are big numbers, indeed!

So, what really matters in the National Football League? Very simply, as stated above - and now to quote the famous Al Davis, the late owner of the Oakland (now Los Vegas) Raiders NFL team - “JUST WIN, BABY, JUST WIN!” Truly -WINNING is the ONLY thing that matters in the world of all high-profile sports, and most intensely, in the National Football League. And without a ‘star’ Super-Bowl caliber NFL quarterback guiding the ship, “JUST WIN, BABY!” although a punchy and clever slogan, most often results in on-the-field disasters - and many ‘goose eggs’ in the win column.

By applying the present disclosure’s artificial intelligence (AI) modalities to the truly colossal list of variables present between NFL prospective quarterbacks, the accuracy of the ultimate decision being the correct one is significantly enhanced.

NFL owners are rightfully and determinedly focused on winning Super Bowls, and anything less is a bitter plum! NFL owners are winners in life with enormous ‘street’ credibility, and they naturally wish to keep right on winning. AI to determine NFL quarterback greatness will prove to be an exceedingly valuable tool to achieve the Holy Grail of Super Bowl victories.

In a first aspect, there is a method for selecting a candidate for a quarterback position of American-style football, the method comprising: determining one or more physical metrics comprising a dimension or a strength or a speed of the candidate; determining one or more skill metrics comprising an agility or a throwing accuracy of the candidate; determining one or more mental metrics comprising an adversity tolerance of the candidate; providing the physical metrics, skill metrics, and mental metrics as inputs to an artificial intelligence model; and determining, as output from the artificial intelligence model, a predicted athletic performance of the candidate in the quarterback position of American-style football.

In another aspect, there is a method for training an artificial intelligence model, the method comprising: using an AI (artificial intelligence) model comprising software configured to determine one or more outputs connected by links to one or more inputs or to one or more internal functions comprising adjustable variables; determining data about each prior player of a plurality of prior players, each prior player comprising an athlete; determining a history of athletic performance of each prior player of the plurality; for each prior player of the plurality: providing the data of the prior player as input to the AI model; determining a predicted athletic performance of the prior player according to output of the AI model; adjusting one or more of the adjustable variables; repeating the above three steps until a predetermined level of agreement is obtained between the predicted athletic performance and the history of athletic performance of the prior player; and providing the AI model to a user, configured to predict a predicted athletic performance of a draft candidate.

In another aspect, there is a method for selecting a particular candidate for a position of quarterback in American-style football, selected from a plurality of candidates, selected according to an artificial intelligence (AI) model, the method comprising: using an AI model trained to predict an athletic performance of each candidate of the plurality, according to measured input data of the candidate; determining two or more performance metrics of each candidate of the plurality; for each candidate of the plurality: providing the performance metrics of the candidate as input to the AI model; determining, as output from the AI model, a predicted athletic performance of the candidate; comparing the predicted athletic performance of all of the candidates of the plurality; and selecting, as the particular candidate, the candidate with a highest predicted athletic performance.

This Summary is provided to introduce a selection of concepts in a simplified form. The concepts are further described in the Detailed Description section. Elements or steps other than those described in this Summary are possible, and no element or step is necessarily required. This Summary is not intended to identify key features or essential features of the claimed subject matter, nor is it intended for use as an aid in determining the scope of the claimed subject matter. The claimed subject matter is not limited to implementations that solve any or all disadvantages noted in any part of this disclosure.

These and other embodiments are described in further detail with reference to the figures and accompanying detailed description as provided below.

### BRIEF DESCRIPTION OF THE DRAWINGS

FIG. 1 is a schematic showing an exemplary embodiment of an AI structure embodied as a neural net, according to some embodiments.

FIGS. 2A-2D are charts showing exemplary embodiments of input data for an AI model, according to some embodiments.

FIG. 2E is a chart showing an exemplary embodiment of output data from an AI model, according to some embodiments.

FIG. 3 is a flowchart showing an exemplary embodiment of a process for training an AI model, according to some embodiments.

FIG. 4 is a flowchart showing an exemplary embodiment of a process for using an AI model, according to some embodiments.

FIG. 5 is a chart showing an exemplary embodiment of the career success trajectory of athletes, according to some embodiments.

Like reference numerals refer to like elements throughout.

### DETAILED DESCRIPTION

Systems and methods disclosed herein (the “systems” and “methods”, also occasionally termed “embodiments” or “arrangements” or “versions” or “examples”, generally according to present principles) can provide urgently needed systems and methods for rationally selecting draft candidates for professional American-style football, as currently practiced in the NFL National Football League in the US. Substantial sums of money are involved in such decisions, especially considering the effect of a good choice on a team’s long-term winning prospects. As with any competitive sport, fans flock to teams that win; hence owners are under enormous pressure to select draft candidates with the greatest promise. However, the selection remains an obscure art with mixed results in the best cases.

An improved selection procedure is disclosed herein based on artificial intelligence (AI) analysis of data on each candidate. The disclosed procedure(s) is/are expected to provide substantially improved selections, resulting in long-term competitive advantages to teams and to the sport in general, according to some embodiments. Most of the examples pertain to selection of a quarterback in American-style football, especially at the level of the National Football League, but the same or similar procedures are expected to enable enhanced selection success for other professional football positions as well, and for other competitive sports, and for other situations requiring superior personal performance under duress (such as the military).

Note: This disclosure is directed not only to the foremost and most renowned American football league - namely, the National Football League (or subsequent name if a name change occurs) - but is also certainly applicable to any league or team playing professional American football, be it in the Unites States or elsewhere.

Disclosed below are some of the multitudinous variables (‘metrics’) which are highly amenable to the application of artificial intelligence analyses to greatly improve the accuracy of determining the athletic ability of a potential NFL player to succeed at the next level up from his collegiate career.

As quarterback is the most difficult position in the NFL to determine with any precision the ultimate ‘winability’ of an athletic prospect - we will use the quarterback position for illustrative purposes - keeping in mind that many of the metrics, such as those below - and others - can certainly be applied to other NFL football positions - to enhance significantly the possibility of a successful selection of a winning NFL athlete.

In examples below, the AI model can take, as input, measurements and facts related to athletic prowess, categorized here into various performance indicators critical for rating or comparing the draft candidates. The AI inputs may be categorized as physical metrics (such as strength), skill metrics (such as passing ability), mental metrics (such as persistence despite setbacks), personal metrics (such as achievements and awards), and institutional metrics (such as high school or college data). A final category, historical metrics, provides a “ground truth” of prior in-game performance data. The historical metrics can be used for training the AI model or to refine the adjustable variables in the AI model to improve accuracy, as in “supervised learning”.

The AI model is developed by adjusting the internal variables until the predicted performance of a particular player, based on his input metrics, is sufficiently in agreement with the observed subsequent performance in, for example, college games. The AI model is further developed by adjusting the variables to accommodate the measurements of a large number of players while predicting their performance with adequate accuracy. Usually a large computer, such as a supercomputer or a quantum computer, is required to process all of the data and all of the players in the large training set, finally arriving at settings of the variables that produce the desired predictions.

In some embodiments, the adjusting of the variables may be performed automatically, such as by a computer. In some embodiments, the variables of the AI model (the “development” model) may be adjusted by another AI model (the “adjuster” model) which has been trained to recognize advantageous adjustment trajectories in complex interconnected data, such as the performance data. The adjuster model may then take over the training process by determining an optimal route, or at least an improved route, for making changes in the development model. In particular, the adjuster model may be able to recognize complex correlations among a large number of inter-related parameters, complex correlations that a human could not possibly comprehend but that enable an economical approach to a successful development model. Absent the AI adjuster model, the supercomputer would have to make a very large number of semi-random iterative adjustment steps before a suitable solution can be reached, if at all. Thus, the AI adjuster model may enhance the optimization of the development model in the same way that the development model, when finished, can enhance the selection of an optimal draft pick.

After being trained, the AI model may be passed to a user for evaluating draft candidates. However, the full development codes may be unwieldy due to the large size and complexity of typical AI models when in development. Alternatively, the AI model may be prepared for use on ordinary computers, by freezing the variables at their optimal settings, identifying and removing inputs that have little or no effect on the predictions, removing internal functions that have little or no effect on the predictions, and otherwise simplifying the prediction-generating code. As a further alternative, the distribution code provided to the end user may be an algorithm or software package or table or matrix or graphical process or weighted sum formula, or other calculation means derived from the AI model, but easier for an ordinary computer to use.

As a further advantageous enhancement, the AI model, or another algorithm that uses the same input data, may determine an uncertainty in each prediction. The uncertainty may be based on the accumulated uncertainties in the input data. The output uncertainty may further account for how “firm” the prediction is (that is, how far the prediction could be altered without significantly reducing its likelihood). When certain pieces of data are unavailable for a particular player, that lack may also be included in the uncertainty calculation. The user may then regard the uncertainty in a prediction as well as the prediction result, and the uncertainty may greatly affect whether the user will decide to act upon the prediction.

In some cases, the AI model may provide, as further output, a comparison or ranking of each candidate for a particular position, or for all of the positions in the sport, based on the input data of each candidate. The user can then review the rankings in determining a selection strategy, based on which positions need to be filled, available funds, and other realities which are outside the domain of the AI model.

In some cases, the user may wish to adapt the AI model to the user’s own priorities or knowledge by adjusting the internal variables of the model in the field. However, adjusting the variables at random would likely spoil the predictive accuracy promptly. Therefore, the supercomputer that developed the AI model may further provide a table of function (the “adjustment matrix”) indicating how the internal variables can be adjusted to obtain a specific result. For example, if the user wishes to emphasize the rushing game instead of the passing game, then the predicted performance of the quarterback candidates would likely be changed. The development computer can thereby assist the user, by determining which variables should be altered, and how much, in order to optimize the predictions for the rushing game, or other customized goals that users may desire.

As used herein, “draft” refers to a process of allocating novice players to various teams, wherein the lowest-scoring team gets first pick of the available candidates. Artificial intelligence (AI) refers to computer-based decision-making according to a multitude of previous examples. An AI “structure” is software configured with numerous internal variables that, when provided with input data, generate an output. The AI structure is “trained” by adjusting the internal variables so that the output agrees with a “ground truth” associated with the input data, in a process termed “supervised machine learning”. (To avoid confusion, the term “training” is reserved herein for AI model development only, and will NOT be used to refer to athletic practicing or skill development.) After the internal variables are suitably trained, the AI structure has thereby graduated to an “AI model” since it is now capable of predicting the future athletic performance of the draft candidates. There is substantial value in determining the most promising candidate; hence the need for the disclosed procedures.

Turning now to the figures, the following examples show how an AI model may be structured, trained, and then used for selecting a draft candidate.

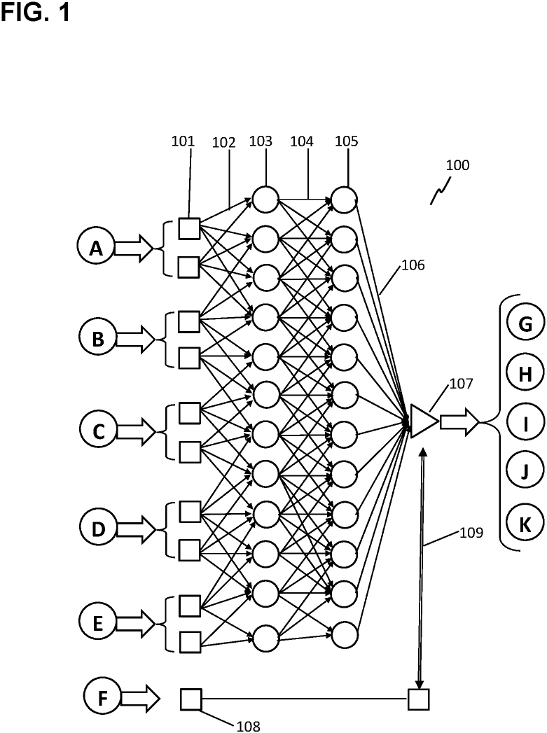

FIG. 1 is a schematic showing an exemplary embodiment of a neural net artificial intelligence model, according to some embodiments. As depicted in this non-limiting example, an AI structure 100, depicted here as a neural net, has a number of inputs 101, one or more intermediate layers of internal functions 103, 105 and one or more outputs 107. The internal functions 103, 105 are operationally connected to the inputs 101 and each other by links 102, 104, while the output 107 is connected to the last layer of the internal functions 105 by further links 106. Although links are shown connecting only a few of the internal functions, in many AI structures each input and each internal function is linked to all of the internal functions of the succeeding layer. All of the links are unidirectional in this case; other embodiments include links going backwards and other complex topologies, although doing so can inhibit convergence.

The AI structure 100 is turned into an AI “model” by adjusting numerous adjustable variables in the internal functions 103, 105. In some embodiments, the links 102, 104, 106 can also include adjustable variables, while other links are simple transfer links. The internal functions can include any type of calculation or logic relating the internal function’s input link values to its output link values. In some embodiments, all of the output link values of a particular internal function are identical, while in other embodiments each link can have a different value and a different relation to that internal function’s input link values.

During model development, the inputs 101 generally include a large number of players of various levels, and the ground truth 108 would include the subsequent performance level of those players in, for example, college or professional football. After each prediction, the output 107 is compared 109 to the actual performance 108 of the player in subsequent games, and unless the prediction is accurate, the internal variables are adjusted again. This cycle is repeated many times with as many players as can be provided with past data and performance, in order to refine the model and improve the prediction accuracy. After the AI model has reached a satisfactory level of predictive accuracy for a sufficient range of players, the model is then rendered suitable for an ordinary computer, as mentioned, and made available to a user. The user than inputs the data about one or a number of draft candidates and determines as output the predicted performance of those candidates. The output 107 may be a prediction of the future athletic performance of each draft candidate separately, or a list or table ranking or comparing a number of draft candidates, depending on user needs. The outputs 107 may further include an uncertainty in the prediction, as mentioned.

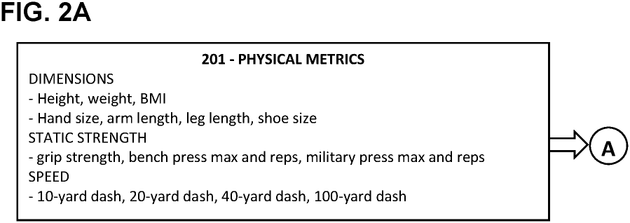

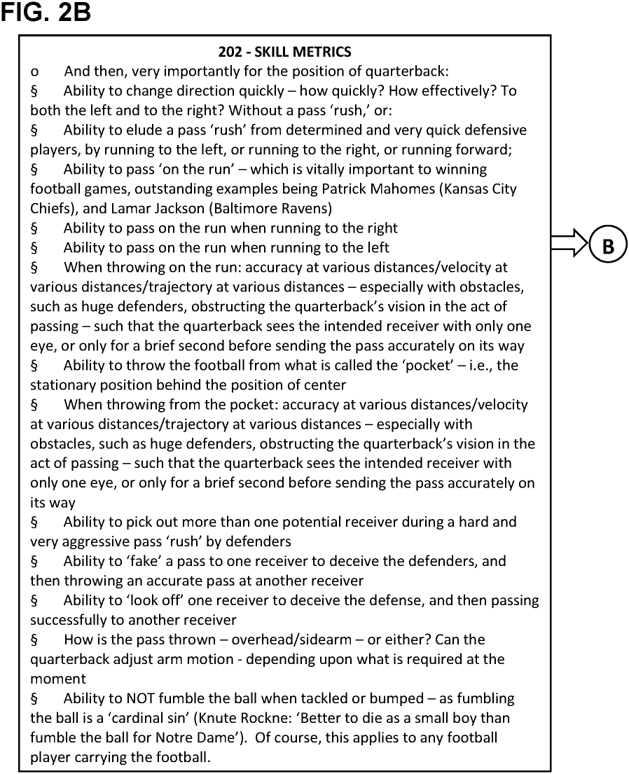

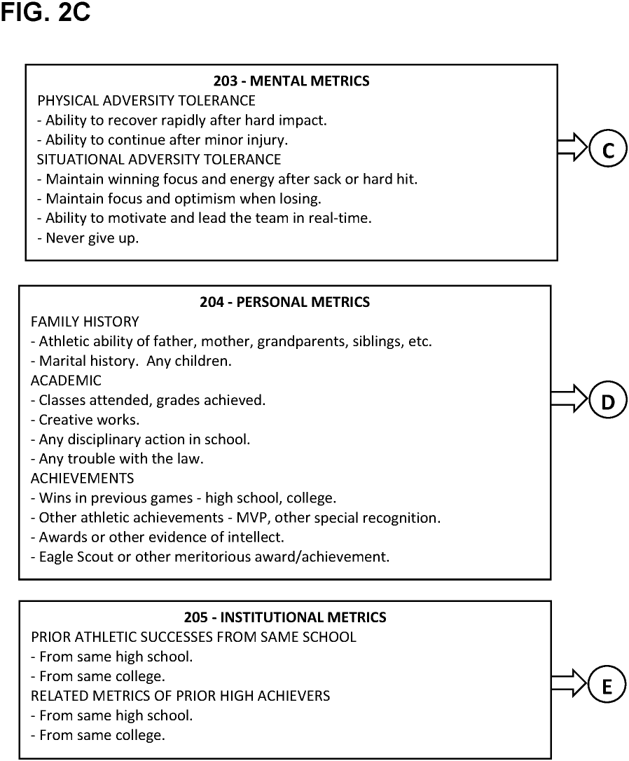

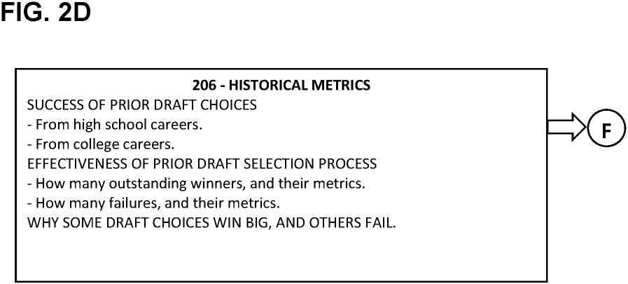

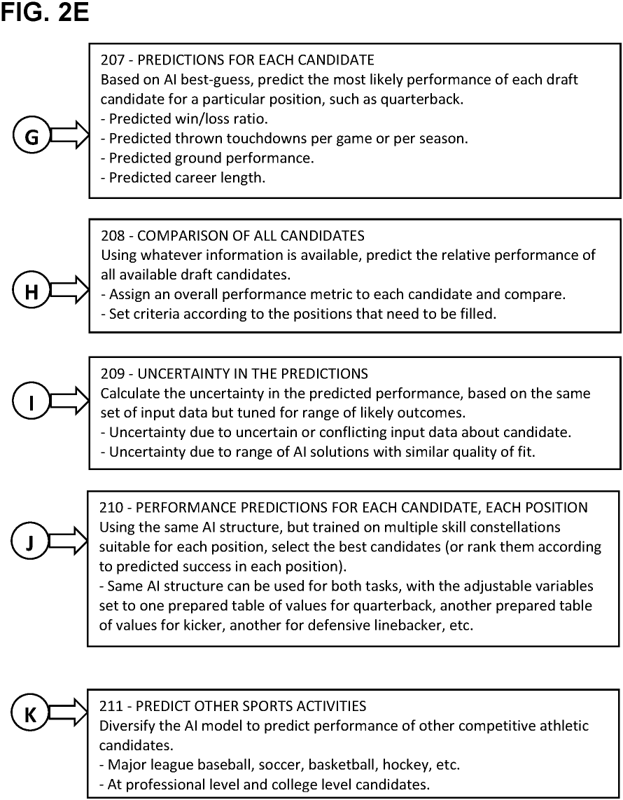

FIG. 2A is a chart showing an exemplary embodiment of input data for an AI model, according to some embodiments. As depicted in this non-limiting example, the inputs may include physical metrics 201 which correspond to item “A” in FIG. 1. The physical metrics 201 may include dimensional data such as the height and weight of a draft candidate, static strength measurements such as the grip strength, and speed measurements such as the 10-yard dash.

FIG. 2B is a chart showing an exemplary embodiment of input data for an AI model, according to some embodiments. As depicted in this non-limiting example, the inputs may further include skill metrics 202, corresponding to item “B” in FIG. 1. The skill metrics 202 may include agility such as the ability to change direction quickly and the ability to pass while in motion, throwing metrics such as accuracy versus distance, versatility such as the ability to throw from all field positions, various passing types such as the ability to fake a pass, and fault avoidance such as the ability to take a hit without dropping the ball.

FIG. 2C is a chart showing an exemplary embodiment of input data for an AI model, according to some embodiments. As depicted in this non-limiting example, the inputs may further include mental metrics 203 corresponding to item “C” in FIG. 1. The mental metrics 203 may include the attitude and self-control required to recover rapidly after a sack, and the self-confidence to maintain a winning focus even when others would give up. These are the critical advantages that make a winning player.

The inputs may include personal metrics 204, corresponding to item “D” in FIG. 1. The personal metrics 204 may include the family history from an athletic perspective, academic history including any disciplinary actions, and special achievements or awards, and especially the candidate’s personal history of game wins and other athletic accomplishments such as MVP (most valuable player) awards and the like.

The inputs may further include institutional metrics 205, corresponding to item “E” in FIG. 1. The institutional metrics 205 may include other players from the same school that went on to achieve great success, as well as the other metrics (physical, skill, etc.) of other players from the same institutions. This accounts for the extraordinary effectiveness of experience at certain institutions.

FIG. 2D is a chart showing an exemplary embodiment of input data for an AI model, according to some embodiments. As depicted in this non-limiting example, the inputs may further include historical metrics 206, corresponding to item “F” in FIG. 1. Unlike the other metrics, the historical metrics 206 are not used as inputs to the AI model directly, but rather as “ground truth” for testing the accuracy of the predictions. The historical metrics 206 may include the success, or otherwise, of prior draft candidates, the effectiveness of prior draft selection processes (with or without AI assistance), and the most difficult question of all: why some candidates win big while others fail. During training of the AI model, the historical metrics 206 are used for supervised training, or to guide the adjustment of the internal variables for increased predictive accuracy.

FIG. 2E is a chart showing an exemplary embodiment of output data from an AI model, according to some embodiments. As depicted in this non-limiting example, the outputs may include predictions for each candidate 207, corresponding to item “G” in FIG. 1. The predictions for each candidate may include the win/loss ratio or the number of thrown touchdowns per game, according to the AI model outputs.

The outputs may further include a comparison 208 of all (or a selected subset) or the candidates. The candidate comparison 208 may include an overall performance metric for each candidate, or a ranking of each candidate regardless of the actual predicted performance, or other comparative analysis of the choices. In practice, the ranking criteria preferably account for the positions that the team needs to fill.

The outputs may further include an uncertainty 209 in the predictions or rankings, corresponding to item “I” in FIG. 1. The uncertainty 209 may be calculated according to uncertainty estimates of the input data, and/or which input data values are unknown or missing, and/or a determination of the range of predictions that have about the same likelihood according to the AI model. If a wide range of different predicted performance levels all have about the same likelihood, as determined by the AI model, then the uncertainty assigned to the prediction must be made at least that wide. In contrast, a prediction with low uncertainty has a rapid decline in likelihood if the prediction value is altered significantly.

The outputs may further include a matrix-like presentation 210 such as an evaluation of the performance of each draft candidate in each position, corresponding to item “J” in FIG. 1. A good player can generally perform well in more than one position. A good player will invoke alternate skill sets when needed, such as when conditions suddenly change (as when a fumble is recovered by the other team, and offense suddenly has to play defense).

The outputs may further include performance predictions of candidates in other sports activities 211, other than American-style football, corresponding to item “K” in FIG. 1. The predictions for other sports activities 211 may include candidates for positions in major-league baseball, soccer, basketball, hockey, and many other competitive athletic positions. Alternatively, the job may be something demanding premium physical performance other than professional sports, such as military or construction or lumberjack positions for example. The AI model, readjusted to account for the strength and skill levels required for the particular occupation, can then be used to select promising candidates in the same way as for professional sports teams. In general, the promise of AI is enhanced decision-making when a multitude of complex, interacting factors are operational.

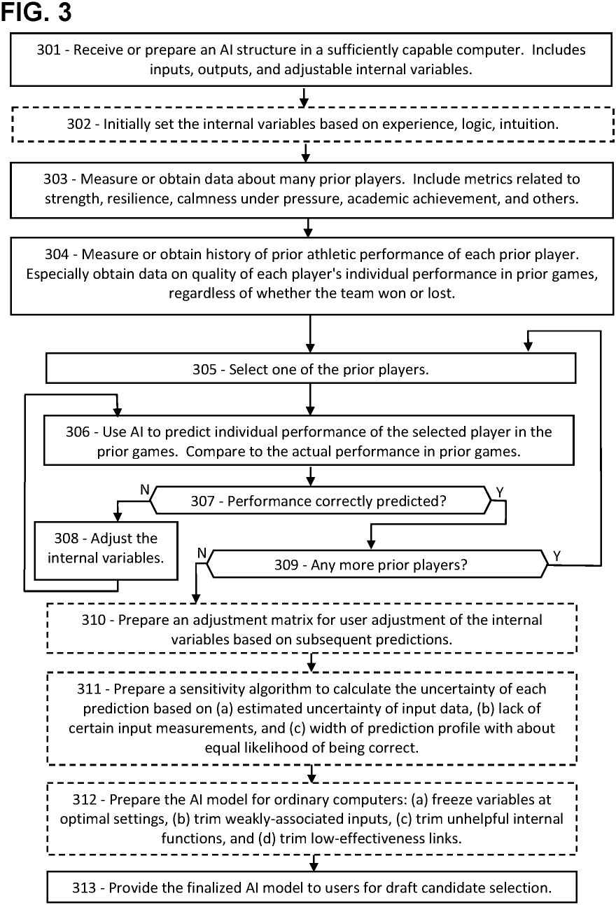

FIG. 3 is a flowchart showing an exemplary embodiment of a process for training an AI model, according to some embodiments. As depicted in this non-limiting example, an AI model for evaluating or predicting athletic performance of players is trained using historical records and past metric data. At 301, an entity can receive and AI structure, or prepare one anew. The AI structure is a computer program generally including inputs, internal variables and functions, and outputs dependent on the internal variables and functions. At 302, the internal variables are set at initial values based on, for example, experience or logic or intuition. Starting with a reasonable set of internal values greatly assists the training and convergence upon a suitable solution capable of accurate predictions.

At 303, input data about a large number of past players is obtained. The data may include the metrics listed an previous figures, or other data related to player performance. At 304, the historical performance of those past players is obtained, such as the number of games won or the player’s individual level of performance in games. This historical data is not used as a direct input to the AI model, but rather as a ground truth for training and adjusting the internal variables.

At 305, one of the past players is selected, perhaps randomly or some other way, and ad 306 the data on that player is fed into the AI as input. The AI then predicts the performance of the player. At 307, the prediction is compared to the historical performance of the player, and if the prediction is incorrect, at 308 the internal variables are adjusted in a way intended to bring the prediction more into alignment with the ground truth. The flow cycles back to 306 for another prediction, until adequate predictive accuracy is obtained. In early stages, a small improvement in predictive accuracy may be sufficient to satisfy this loop, whereas in later stages of fine-tuning, a much higher accuracy may be required as the AI model improves.

After a sufficiently accurate prediction is obtained, if there are additional past players in the training set at 309, the flow cycles back to 305 to refine the model using each of the past players in turn.

After all of the players have been used for input training, the list of past players may be used as input again, and the overall cycle may be repeated for all of the past players repeatedly, until the model is finally configured to provide adequate predictions for all of them, or until further improvement in prediction cannot be obtained. For clarity, this loop is not explicitly shown, but is to be understood from the 305 to 309 cycles.

As an alternative, the AI model can be trained using accumulated or averaged data, in which several of the past players with similar features and similar performance histories can be averaged together, thereby enabling a general solution be be reached faster than using the individual players separately. Many other strategies for training AI models using data agglomeration and iterative cycling are known and are included in the disclosure.

At 310, the finalized AI model may be used to develop an adjustment matrix, which indicates how the internal variables can be altered in order to obtain a desired change in the output predictions. For example, if a particular user is interested in using the model to select an outstanding defensive lineman rather than an offensive position, the adjustment matrix may indicate which of the internal variables to change, and in what direction, and how much. Since it is generally easier for the AI model developer to measure such cause-effect relationships, and to prepare the corresponding adjustment matrix for common types of user priorities, the adjustment matrix may be provided to the user along with the AI model itself, for field adjustment as needed.

At 311, the AI model, or another AI model, may be trained to calculate the uncertainty in the predictions. The uncertainty is valuable so that the user can determine whether to trust and act upon the prediction, or to disregard the prediction if the uncertainty is large. The uncertainty may be based on the uncertainties of the input values, if known or can be guessed. If some pieces of input data are missing or inconsistent with other pieces, the uncertainty in the prediction must be increased to reflect that lack. In addition, the AI model can determine a range of predictions that all have about the same likelihood, based on the inputs. If that range is quite narrow, then the uncertainty is low and the prediction may be considered precise. If that range is broad, then the prediction uncertainty is correspondingly large. Users may understand that it is risky to trust a software prediction at face value, without checking the uncertainty or despite a large reported uncertainty.

At 312, the AI model may be adapted for use by a user in an ordinary computer, instead of the supercomputer that the developer likely employed. Adapting the trained AI model for field use may include freezing the internal variables at their optimal values, based on the set of past players that the AI model was trained on; unimportant inputs may be eliminated (depending on the particular application/sport/position desired); unhelpful internal functions, having little or no effect on the output, may be eliminated; and links that are either redundant or irrelevant to the output can be trimmed, thereby providing a lighter, leaner software package that may be easier for users.

At 313, the finalized AI model, or the trimmed version, or an algorithm or software derived from it, is then provided to users for rationally selecting draft candidates for competitive sports.

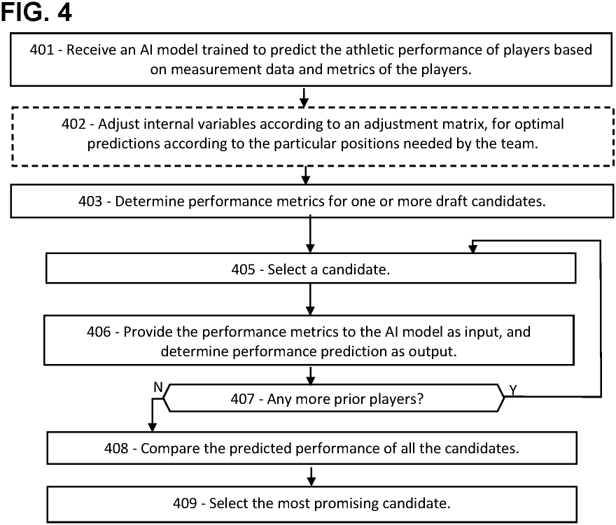

FIG. 4 is a flowchart showing an exemplary embodiment of a process for using an AI model, according to some embodiments. As depicted in this non-limiting example, at 401 a user receives an AI model trained to predict the athletic performance of a player based on the metrics data as described above. At 402, optionally, the user may adjust the internal variables of the AI model, using an adjustment matrix provided by the developer of the AI model, to optimize the predictions for the positions (or sports or weather conditions or other criterion) desired by the user.

At 403, the user determines or otherwise obtains the input data about the draft candidates, or at least a subset of the draft candidates that the user intends to consider. At 405, one candidate is selected and at 406, the input data on that candidate are provided to the AI model. The predicted performance of the candidate is then determined by the AI model. At 407, the user repeats the above cycle for all of the candidates of interest, and at 408 the user compares the predicted performance of the candidates, enabling the best choice to be made at 409.

Alternatively, not shown, the input data on all of the candidates may be provided to the AI model, and the AI model may be configured to rank the candidates or otherwise perform the comparison step based on the data.

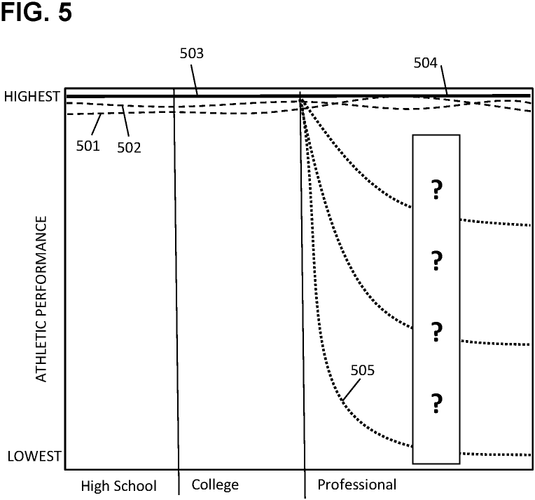

FIG. 5 is a chart showing an exemplary embodiment of a history of athletic performance, according to some embodiments. As depicted in this non-limiting, highly schematic representation, the athletic performance of players can be reliably predicted in some sports but not others, according to prior art. The athletic performance (such as win/loss ratio) of a player is shown on the vertical scale from lowest to highest performance levels, while the career stage (high school, college, professional) is shown horizontally. Dotted lines 501, 502 indicate the likely performance trajectory of top athletes in sports such as baseball 501 and tennis 502, as well as golf and many other sports. As depicted, an athlete who exhibits top performance in high school and college will usually continue to provide top performance as a professional.

In contrast, the quarterback position is much more difficult to predict, based solely on pre-professional scoring. The solid line 503 shows an athlete exhibiting top performance as a quarterback in high school and college. In some cases 504, the athlete continues to excel as a NFL professional as shown at 504. In other cases 505, the top performing quarterbacks in high school and college fail in the professional league. The historical record shows that it is difficult to discriminate between the quarterback candidates that continue to perform professionally 504, and those who fail to perform 505 in professional football, unlike the other sports.

Hence, the need for an improved means for determining which candidate has the best chance of prevailing as an NFL quarterback.

AI models tend to be most adept at solving problems that are highly complex, with multiple interacting or correlated parameters and highly nonlinear effects. In the context of athletic performance prediction, AI may contribute beneficially in selecting which draft candidate best meets the needs of a particular team.

The AI model embodiments of this disclosure may be aptly suited for cloud backup protection, according to some embodiments. Furthermore, the cloud backup can be provided cyber-security, such as blockchain, to lock or protect data, thereby preventing malevolent actors from making changes.

In some embodiments, non-transitory computer-readable media may include instructions that, when executed by a computing environment, cause a method to be performed, the method according to the principles disclosed herein. In some embodiments, the instructions (such as software or firmware) may be upgradable or updatable, to provide additional capabilities and/or to fix errors and/or to remove security vulnerabilities, among many other reasons for updating software. In some embodiments, the updates may be provided monthly, quarterly, annually, every 2 or 3 or 4 years, or upon other interval, or at the convenience of the owner, for example. In some embodiments, the updates (especially updates providing added capabilities) may be provided on a fee basis. The intent of the updates may be to cause the updated software to perform better than previously, and to thereby provide additional user satisfaction.

The systems and methods may be fully implemented in any number of computing devices. Typically, instructions are laid out on computer readable media, generally non-transitory, and these instructions are sufficient to allow a processor in the computing device to implement the method of the invention. The computer readable medium may be a hard drive or solid state storage having instructions that, when run, or sooner, are loaded into random access memory. Inputs to the application, e.g., from the plurality of users or from any one user, may be by any number of appropriate computer input devices. For example, users may employ vehicular controls, as well as a keyboard, mouse, touchscreen, joystick, trackpad, other pointing device, or any other such computer input device to input data relevant to the calculations. Data may also be input by way of one or more sensors on the robot, an inserted memory chip, hard drive, flash drives, flash memory, optical media, magnetic media, or any other type of file -storing medium. The outputs may be delivered to a user by way of signals transmitted to robot steering and throttle controls, a video graphics card or integrated graphics chipset coupled to a display that maybe seen by a user. Given this teaching, any number of other tangible outputs will also be understood to be contemplated by the invention. For example, outputs may be stored on a memory chip, hard drive, flash drives, flash memory, optical media, magnetic media, or any other type of output. It should also be noted that the invention may be implemented on any number of different types of computing devices, e.g., embedded systems and processors, personal computers, laptop computers, notebook computers, net book computers, handheld computers, personal digital assistants, mobile phones, smart phones, tablet computers, and also on devices specifically designed for these purpose. In one implementation, a user of a smart phone or Wi-Fi-connected device downloads a copy of the application to their device from a server using a wireless Internet connection. An appropriate authentication procedure and secure transaction process may provide for payment to be made to the seller. The application may download over the mobile connection, or over the Wi-Fi or other wireless network connection. The application may then be run by the user. Such a networked system may provide a suitable computing environment for an implementation in which a plurality of users provide separate inputs to the system and method.

It is to be understood that the foregoing description is not a definition of the invention but is a description of one or more preferred exemplary embodiments of the invention. The invention is not limited to the particular embodiments(s) disclosed herein, but rather is defined solely by the claims below. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. For example, the specific combination and order of steps is just one possibility, as the present method may include a combination of steps that has fewer, greater, or different steps than that shown here. All such other embodiments, changes, and modifications are intended to come within the scope of the appended claims.

As used in this specification and claims, the terms “for example”, “e.g.”, “for instance”, “such as”, and “like” and the terms “comprising”, “having”, “including”, and their other verb forms, when used in conjunction with a listing of one or more components or other items, are each to be construed as open-ended, meaning that the listing is not to be considered as excluding other additional components or items. Other terms are to be construed using their broadest reasonable meaning unless they are used in a context that requires a different interpretation.

The present disclosure, employing extremely sensitive artificial intelligence modalities to analyze the gigantic number of physical, mental, and other intangible metrics - as well as analyzing all available background information -provides a superior methodology to answer this all-important question: Which athlete will most likely succeed in the National Football League, and which athlete most likely will not.

1. A method for selecting a candidate for a quarterback position of American-style football, the method comprising:
a) determining one or more physical metrics comprising a dimension or a strength or a speed of the candidate;
b) determining one or more skill metrics comprising an agility or a throwing accuracy of the candidate;
c) determining one or more mental metrics comprising an adversity tolerance of the candidate;
d) providing the physical metrics, skill metrics, and mental metrics as inputs to an artificial intelligence model; and
e) determining, as output from the artificial intelligence model, a predicted athletic performance of the candidate in the quarterback position of American-style football.

2. The method of claim 1, further comprising:
a) determining one or more personal metrics comprising an athletic ability of a relative of the candidate or an academic accomplishment of the candidate;
b) determining one or more institutional metrics comprising an athletic record of a high school or college attended by the candidate; and
c) providing the personal and institutional metrics as further inputs to the artificial intelligence model.

3. The method of claim 1, further comprising:
a) providing, as further input to the artificial intelligence model, data about two or more further candidates; and
b) determining, as further output from the artificial intelligence model, a ranking of predicted athletic performance of the candidate in comparison with the further candidates.

4. The method of claim 1, further comprising determining, as further output from the artificial intelligence model, an uncertainty in the predicted athletic performance of the candidate.

5. The method of claim 1, wherein the physical metrics further comprise a time for the candidate to run a ten-yard dash.

6. The method of claim 1, wherein the skill metrics further comprise an accuracy of throwing while in motion.

7. The method of claim 1, wherein the mental metrics further comprise an ability to maintain a particular level of athletic performance while having a lower score than an opposing team.

8. A method for training an artificial intelligence model, the method comprising:
a) using an AI (artificial intelligence) model comprising software configured to determine one or more outputs connected by links to one or more inputs or to one or more internal functions comprising adjustable variables;
b) determining data about each prior player of a plurality of prior players, each prior player comprising an athlete;
c) determining a history of athletic performance of each prior player of the plurality;
d) for each prior player of the plurality:
i) providing the data of the prior player as input to the AI model;
ii) determining a predicted athletic performance of the prior player according to output of the AI model;
iii) adjusting one or more of the adjustable variables;
iv) repeating the above three steps until a predetermined level of agreement is obtained between the predicted athletic performance and the history of athletic performance of the prior player; and
e) providing the AI model to a user, configured to predict a predicted athletic performance of a draft candidate.

9. The method of claim 8, further comprising, before providing the data about each prior player as input to the AI model, setting each of the adjustable variables at an initial setting according to an opinion of a person responsible for evaluating athletic performance of athletes.

10. The method of claim 8, wherein the predetermined level of agreement comprises agreement, within a predetermined range, of a win/loss ratio or of a number of thrown touchdowns per game.

11. The method of claim 8, further comprising preparing an adjustment matrix comprising instructions for adjusting each of the adjustable variables according to a predetermined objective.

12. The method of claim 8, further comprising determining an uncertainty of each prediction of athletic performance.

13. The method of claim 8, further comprising preparing the AI model to be operational in a computer of a user.

14. The method of claim 13, wherein the preparing the AI model to be operational in a computer of a user comprises:
a) freezing or maintaining constant each of the adjustable variables;
b) trimming or removing each input determined to have a low association with the output;
c) trimming or removing each internal function determined to have low association with the output; and
d) trimming or removing each link determined to have low association with the output.

15. A method for selecting a particular candidate for a position of quarterback in American-style football, selected from a plurality of candidates, selected according to an artificial intelligence (AI) model, the method comprising:
a) using an AI model trained to predict an athletic performance of each candidate of the plurality, according to measured input data of the candidate;
b) determining two or more performance metrics of each candidate of the plurality;
c) for each candidate of the plurality:
i) providing the performance metrics of the candidate as input to the AI model;
ii) determining, as output from the AI model, a predicted athletic performance of the candidate;
d) comparing the predicted athletic performance of all of the candidates of the plurality; and
e) selecting, as the particular candidate, the candidate with a highest predicted athletic performance.

16. The method of claim 15, wherein the AI model is trained using performance metrics of prior players other than the candidates of the plurality.

17. The method of claim 15, wherein the input data of each candidate comprises at least two of:
a) a determination of strength;
b) a determination of speed;
c) a determination of throwing accuracy; and
d) a history of athletic achievements.

18. The method of claim 15, wherein the predicted athletic performance of each candidate comprises:
a) a predicted win/loss ratio; and
b) a predicted number of thrown touchdowns per game or per season.

19. The method of claim 15, wherein the selecting the candidate with a highest predicted athletic performance comprises comparing or ranking all the candidates of the plurality according to an expected number of wins per season, and selecting, as the particular candidate, the candidate with a highest number of expected wins per season.

20. The method of claim 15, further comprising determining, for each candidate, an uncertainty in the predicted athletic performance of each candidate of the plurality.
